(19) United States
(12) Patent Application Publication
(10) Pub. No.: US 2024/0268760 A1
(43) Pub. Date: Aug. 15, 2024

(54) SENSOR INSERTION

(71) Applicant: PercuSense, Inc., VALENCIA, CA (US)

(72) Inventors: KATHERINE WOLFE, DUNWOODY, GA (US); BENJAMIN J PAGLIUSO, MARINA DEL REY, CA (US); BELLA T NAVA, LOS ANGELES, CA (US)

(73) Assignee: PercuSense, Inc., VALENCIA, CA (US)

(21) Appl. No.: 18/108,500

(22) Filed: Feb. 10, 2023

(51) Int. Cl. A61B 5/00

(52) U.S. Cl. CPC A61B 5/6833; A61B 5/6848

(57) ABSTRACT

In one embodiment, an insertion assembly is disclosed that includes an electronics assembly. The electronics assembly includes a base and a housing and the housing has an enclosed portion and a mating portion. The insertion assembly further includes a sensor assembly that is detached from the electronics assembly. The sensor assembly includes a sensor and at least one mating feature. The sensor assembly is detachably coupled to a slider in a first position. The slider is coupled to an energy storage system. When the slider transitions to a second position, the sensor assembly is coupled to the electronics assembly to define an on-body assembly. The transition to the second position loads the energy storage system and upon actuation, the energy storage system is unloaded to transition the slider to the first position and the on-body assembly is positioned to decouple from the slider.

### FIELD OF THE INVENTION

The present invention is generally directed to devices and methods that perform in vivo monitoring of an analyte or analytes such as, but not limited to glucose, lactate and oxygen. In particular, the devices and methods are for the insertion and activation of electrochemical sensors that provide information regarding the presence or amount of an analyte or analytes within a subject.

### BACKGROUND OF THE INVENTION

In vivo monitoring of particular analytes can be critically important to short-term and long-term well being. For example, the monitoring of glucose can be particularly important for people with diabetes in order to determine insulin or glucose requirements. In another example, the monitoring of lactate in postoperative patients can provide critical information regarding the detection and treatment of sepsis.

The need to perform continuous or near continuous analyte monitoring has resulted in the development of a variety of devices and methods. Some methods place electrochemical sensor devices designed to detect the desired analyte in blood vessels while other methods place the devices in subcutaneous or interstitial fluid.

For sensors placed in subcutaneous or interstitial fluid it may be critical or at least desirable to place the sensor at a preferred depth in order to obtain preferred sensor performance while also maximizing the useful life of the implanted sensor. Sensors not inserted at a desired depth may have decreased life expectancy or have spurious noise introduced into the sensor signal.

Challenges associated with inserting in-vivo sensors include, but are not limited to, consistently placing or inserting the sensor at a preferred depth within a subject and ensuring the sensor is supported throughout the insertion process to avoid deformation of the sensor such as bending or kinking. What is needed are insertion techniques that enable insertion of implantable sensors.

### BRIEF SUMMARY OF THE INVENTION

In one embodiment, an insertion assembly is disclosed that includes an electronics assembly. The electronics assembly includes a base and a housing and the housing has an enclosed portion and a mating portion. The insertion assembly further includes a sensor assembly that is detached from the electronics assembly. The sensor assembly includes a sensor and at least one mating feature. The sensor assembly is detachably coupled to a slider in a first position. The slider is coupled to an energy storage system. When the slider transitions to a second position, the sensor assembly is coupled to the electronics assembly to define an on-body assembly. The transition to the second position loads the energy storage system and upon actuation, the energy storage system is unloaded to transition the slider to the first position and the on-body assembly is positioned to decouple from the slider.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1A is a pseudo-isometric view of an insertion assembly, in accordance with embodiments of the present invention.

FIG. 1B is a side view of the insertion device and the on body assembly separated from each other.

FIG. 2 is an exploded pseudo-isometric view of the insertion device in accordance with embodiments of the present invention.

FIG. 3A is a pseudo-isometric view of the on body assembly, in accordance with embodiments of the present invention.

FIGS. 3B-3C, are different illustrations of the on body assembly where the sensor assembly 306 is detached from the electronic assembly, in accordance with embodiments of the present invention.

FIG. 4 is an exemplary pseudo-isometric exploded view of the electronics assembly, in accordance with embodiments of the present invention.

FIG. 5A is an exemplary pseudo-isometric exploded view of the sensor assembly, in accordance with embodiments of the present invention.

FIGS. 5B and 5C are exemplary illustrations of select components within the sensor assembly that illustrate the interaction between the sensor and features of the cap, in accordance with embodiments of the present invention.

FIG. 6 is an exemplary illustration of the insertion device, an electronics assembly (without the patch for clarity) and a sensor assembly positioned relative to each other in a first position that is prior to the sensor assembly being coupled to the electronics assembly, in accordance with embodiments of the present invention.

FIG. 7A is a pseudo-isometric view of an insertion assembly just after coupling the sensor assembly to the electronics assembly (without the patch for clarity), in accordance with embodiments of the present invention.

FIG. 7B is a cross-sectional view of the insertion assembly looking at a section defined by section line A-A in FIG. 7A, in accordance with embodiments of the present invention.

FIG. 8 is a cross-section view of the partial insertion assembly in the second position, in accordance with embodiments of the present invention.

FIG. 9 is a cross-section view of the partial insertion assembly after the slider has returned to the first position and the on body assembly has been detached from the slider, in accordance with embodiments of the present invention.

### DETAILED DESCRIPTION

Sensor insertion techniques are often heavily influenced by the techniques used to manufacture the sensor. For example, constructing a sensor on a rigid substrate may require the sensor to be protected within a needle to prevent unintentional flexing or bending. With a sensor designed to be made on a flexible substrate, insertion techniques can be employed that simplify insertion, while reducing both the on-body footprint and overall volume of the insertion system.

In many embodiments, additional features or elements can be included or added to the exemplary features described above. Alternatively, in other embodiments, fewer features or elements can be included or removed from the exemplary features described above. In still other embodiments, where possible, combination of elements or features discussed or disclosed incongruously may be combined together in a single embodiment rather than discreetly as in the exemplary discussion. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

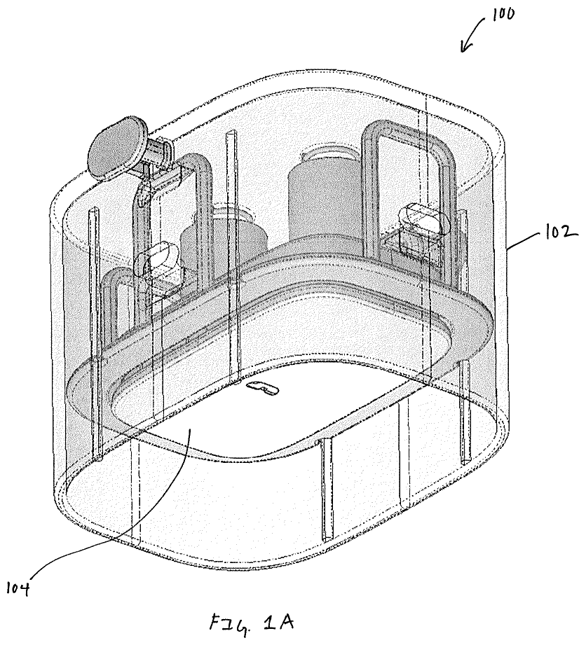

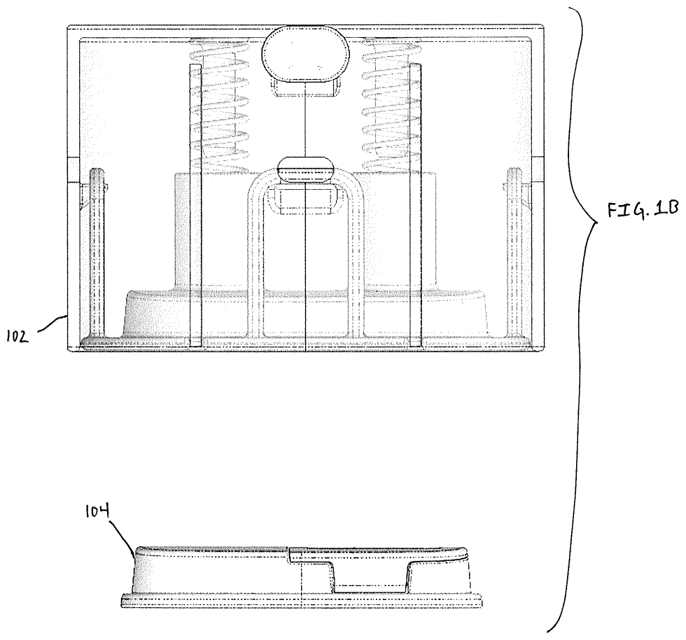

FIG. 1A is a pseudo-isometric view of an insertion assembly 100, in accordance with embodiments of the present invention. The insertion assembly 100 shown in FIG. 1A includes a portion of an insertion device 102 and an on body assembly 104. The insertion device 102 is illustrated as being partially transparent to show various elements within the insertion device 102 along with the on body assembly 104 within the insertion device 102.

FIG. 1B is a side view of a portion of the insertion device 102 and the on body assembly 104 separated from each other.

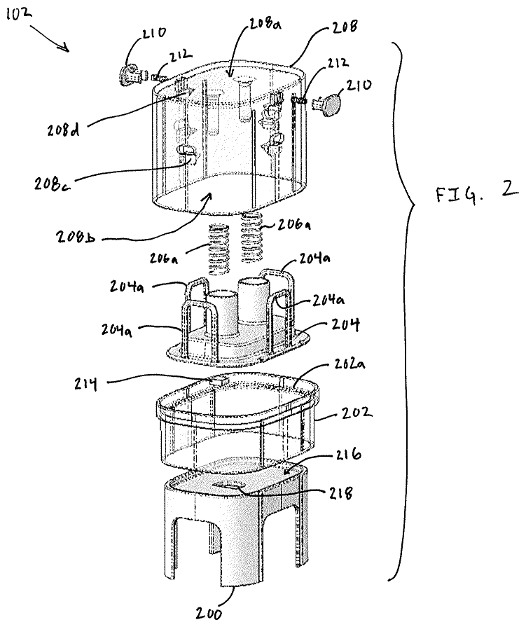

FIG. 2 is an exploded pseudo-isometric view of the insertion device 102 in accordance with embodiments of the present invention. The insertion device 102 includes a stand 200, a skirt 202, a slider 204, energy storage 206a, an encasement 208, along with actuators 210. In some embodiments, the actuators 210 further include springs 212. A slider engagement 214 is coupled to the slider 204. In some embodiments, the slider engagement 214 is a magnet. Alternatively, in other embodiments, the slider engagement 214 is a ferrous metal that may be coupled to a magnet. More generally, the slider engagement 214 may be considered part of a coupling that utilizes mechanical retaining forces.

In many embodiments, the energy storage 206a are compression springs. While the embodiments illustrated throughout this paper include a plurality of energy storage 206a, the use of fewer or additional springs or alternative types of energy storage should be considered within the scope of this disclosure. Examples of alternative types of energy storage include, but are not limited to different types of springs, elastomers, bands, compressed fluids and the like.

The encasement 208 includes an encasement top 208a that defines an encasement interior 208b. Within the encasement interior 208b is at least a first slider stop 208c and at least a second slider stop 208d. FIG. 2 includes a first plurality of first slider stops 208c and a second plurality of second slider stops 208d. However, in other embodiments, additional or fewer first slider stops 208c and second slider stop 208d may be used. The first slider stop 208c and second slider stop 208d are intended to interact with a slider retainer 204a.

When the slider retainer 204a is engaged with the first slider stop 208c, the slider 204 is located in a first position. In the first position, the energy storage 206a are in a first position that defines a minimal energy storage condition. In embodiments where the energy storage 206a is a spring, the first position results in no, or minimal compression, of the energy storage 206a. When the slider retainer 204a is engaged with the second slider stop 208d, the slider 204 is located in a second position. In the second position, the energy storage 206a are storing energy that when released, will return the slider 204 to the first position. As illustrated in FIG. 2, when the slider retainer 204a engages the second slider stop 208d, the energy storage 206a is compressed.

In many embodiments, when the slider 204 is in the second position, an actuator 210 is used to displace slider retainers 204a from the second slide stop 208d. The disengagement of the slider retainers 204a from the second slide stop 208d releases the energy in energy storage 206a resulting in the slider 204 returning to the first position where the slider retainers 204a are engaged with the first slider stop 208c.

The skirt 202 includes an encasement lip 202a. The encasement lip 202a is intended to interface with an encasement bottom 208c. Additionally, the skirt 202 is configured to slide over the stand 200. The stand 200 includes a depression 216 that is configured to help locate components associated with the on body assembly 104 (FIG. 1A). As illustrated in FIG. 2, the stand 200 further includes a first stand aperture 218 that also locates and aligns components or elements associated with the on body assembly 104 (FIG. 1A)

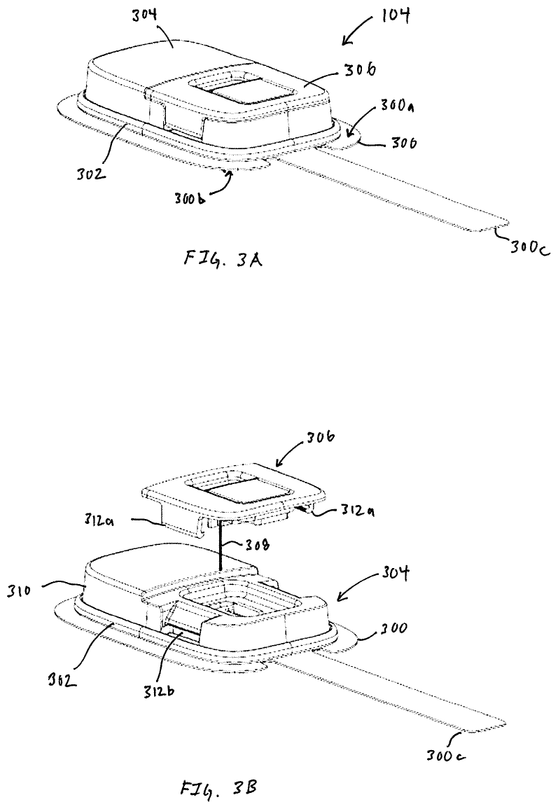

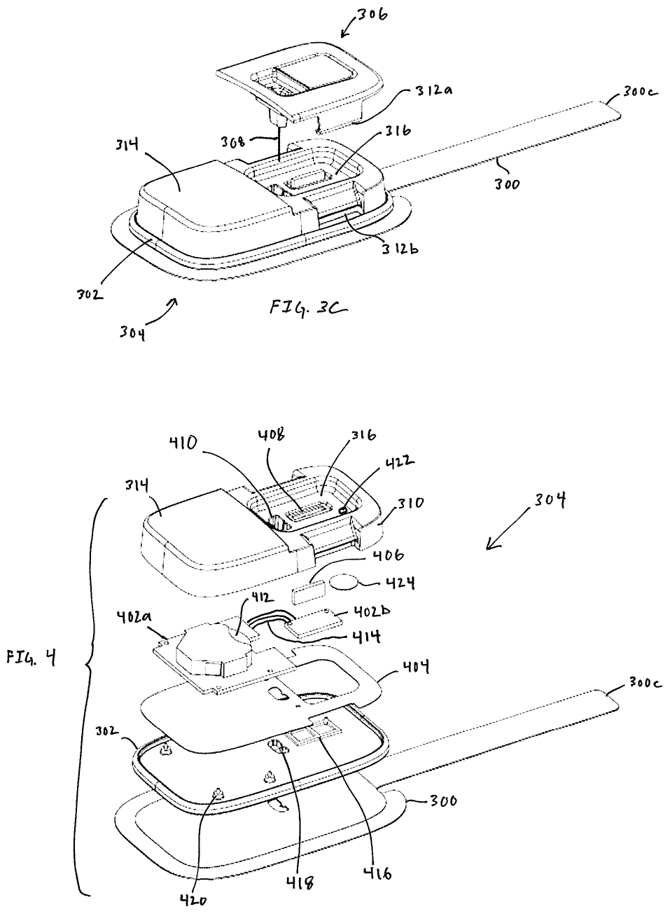

FIG. 3A is a pseudo-isometric view of the on body assembly 104, in accordance with embodiments of the present invention. The on body assembly 104 includes a patch 300, a base 302, an electronic assembly 304 and a sensor assembly 306. In many embodiments, a first side 300a of the patch 300 is coupled to the base 302 using a mechanical coupling such as, but not limited to, an adhesive. A second side 300b of the patch 300 is intended to be attached or coupled to a subject (not shown) using an adhesive for a specified minimum period of time. In some embodiments the minimum period of time may be as short as a few hours. While in other embodiments, the minimum period of time may be multiple days or even multiple weeks. In preferred embodiments, the second side 300b is covered by a release liner (not shown) that needs to be removed to expose the adhesive on the second side 300b.

FIGS. 3B-3C, are different illustrations of the on body assembly 104 where the sensor assembly 306 is detached from the electronic assembly 304, in accordance with embodiments of the present invention. These views show various other features of both the sensor assembly 306 and the electronic assembly 304. For example, The sensor assembly 306 includes a housing 301, a mating feature A 312a and the electronics assembly 304 includes a mating feature B 312b. As illustrated, the mating feature A 312a is a snap while the mating feature B 312b is configured to receive and retain the mating feature A 312a. The embodiments illustrated should not be construed as limiting as other embodiments can couple the sensor assembly 306 to the electronics assembly 304 using other techniques such as, but not limited to mechanical couplings like adhesives, fasteners, pins, and joinery. In preferred embodiments, whatever technique is used to couple the mating feature A 312a with the mating feature B 312b, it may be preferable to minimize the likelihood of uncoupling the sensor assembly 308 from the electronics assembly 304 as that may compromise the integrity of interior seals within the on body assembly 104.

Though described above as having a single mating feature A 312a and mating feature B 312b, many embodiments can include a plurality of the mating feature A 312a and a corresponding plurality of mating feature B 312b. For example, as illustrated in FIGS. 3B and 3C, the sensor assembly 306 includes multiple mating features 312a that are clips. Similarly, the electronics assembly 304 includes a corresponding number of mating features 312b to accept the clips from the sensor assembly 306. In other embodiments having multiple mating features different types of mating features may be used such as a single clip used with an adhesive or an adhesive used with a screw or various combinations thereof.

In FIG. 3C the housing 310 is shown as having a receiver portion 316 and an enclosed portion 314. The receiver portion 316 is illustrated as being a recess or depression that accommodates the sensor assembly 306. The enclosed portion 314 encapsulates components between the housing 310 and the base 302. FIG. 3C also includes the patch 300 that is sized to fit within the depression 216 (FIG. 2) and assist or enable alignment of the electronic assembly 304 with the stand 200. Additionally, the patch 300 includes a tail 300c that is configured to interact with the stand 200 (FIG. 2 and FIG. 7B) to provide additional alignment of the electronics assembly 302 with the stand 200.

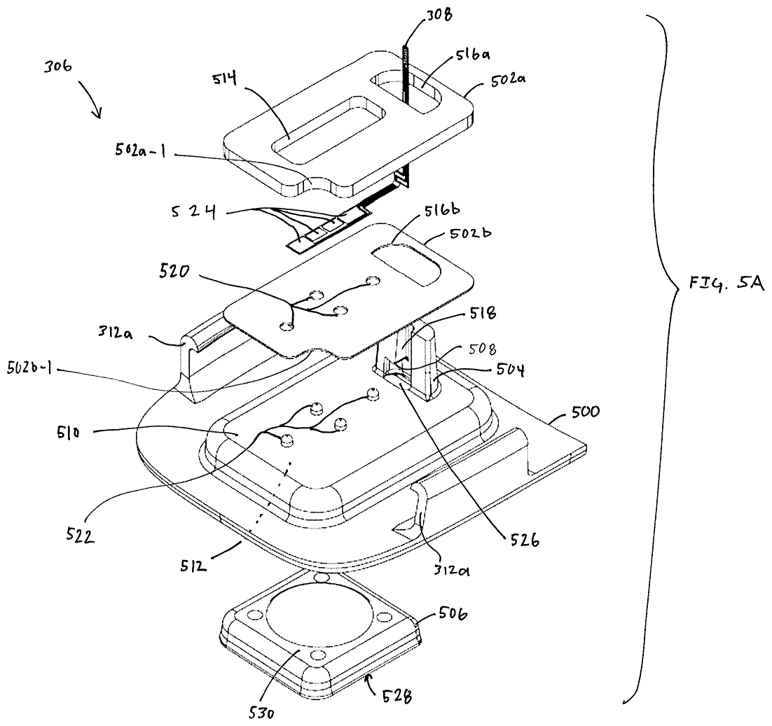

FIG. 4 is an exemplary pseudo-isometric exploded view of the electronics assembly 304, in accordance with embodiments of the present invention. This illustration includes exemplary elements that may be found between the housing 310 and the base 302 of the electronics assembly 304. For example, in many embodiments, a gasket 404, printed circuit boards (PCBs) 402a and 402b, along with conductor 406 and barrier 424 are all located between the housing 310 and the base 302. Note that the gasket 404 is merely an exemplary embodiment and other embodiments may utilize different sealing or waterproofing techniques, such as, but not limited to adhesives or physical bonding such as ultrasonic welding or heat staking. While the embodiment illustrated in FIG. 4 includes a plurality of PCBs (402a and 402b), other embodiments may utilize a single PCB. FIG. 4 further includes additional features of the housing 310 such as a contact aperture 408 and a sharp support aperture 410.

The conductor 406 is intended to make electrical contact with the PCB 402b. Additionally, the conductor 406 is also intended to be exposed within the receiver portion 316 by being placed through the contact aperture 408. The embodiment illustrated in FIG. 4 should not be construed as limiting. In other embodiments, alternate techniques can be used to pass the conductor 406 into the receiver portion 316. For example, in some embodiments electrical conductors can be molded into or through the housing 310. In such embodiments, there may be plurality of contact apertures that enable a conductor such as conductive pins, wires or the like, to make electrical contact with a PCB while being exposed within the receiver portion 316. In many embodiments, the barrier 424 is a moisture barrier and it is positioned below a vent 422 located in the receiver portion 316.

In many embodiments, the PCB 402a is intended to be fully contained within the enclosed portion 314 of the housing 310. As illustrated, the PCB 402a may include a power supply 412. In preferred embodiments, the power supply 412 is a battery. However in alternative embodiments, the power supply 412 may be any energy storage device such as, but not limited to solar cells, capacitors, fuel cells or the like. In many embodiments, the PCB 402a is electrically coupled with the PCB 402b using connection 414. In some embodiments the connection 414 is a flex circuit material while in other embodiments a wire ribbon or individual wires are used to electrically connect PCB 402a and PCB 402b. The use of two PCBs 402a and 402b should not be construed as limiting. Rather, in alternative embodiments, a single PCB or additional PCBs may be used while still being within the scope of this disclosure.

The base 302 includes a support feature 416, base aperture 418 and at least one locating feature 420. The support feature 416 is intended to support the PCB 402b. In many embodiments, the support feature 416 also includes one or more locating features that interface with the PCB 402b such as, but not limited to a post, column, ridge, groove or the like. The base aperture 418 is an opening that passes through the base 302. The base aperture 418 is intended to be axially aligned with the sharp support aperture 410. The at least one locating feature 420 is intended to assist in aligning at least one other element within the electronics assembly 304 the the base 302. For example, the locating feature 420 may assist in aligning the gasket 404 with the base 320. In other embodiments, the locating feature 420 may assist in aligning a plurality of elements within the electronics assembly 304 with the base 302. For example, the locating feature 410 may assist in aligning the gasket 404 and the PCB 402a with the base 302. In other embodiments, the base 302 may have a plurality of locating features where each of the plurality of locating features is configured to assist in aligning one or more additional elements within the electronics assembly 304.

For example, in some embodiments, the base 302 may include locating features that assist in aligning the PCB 402b. In some of these embodiments, locating features to assist in placement of the PCB 402b may be part of, or incorporated into, the support feature 416. In other embodiments the base 302 may include locating features that assist in aligning or placing the PCB 402a relative to the base 302.

The gasket 404 is intended to provide a seal between the base 302 and the housing 310. In preferred embodiments the gasket 404 is sized or configured to produce an interference fit between the base 302 and the housing 310. In many embodiments the interference fit takes the form of compression between the base 302 and the housing 310. Accordingly, the gasket 404 may be made from a flexible material. Preferred materials include, but are not limited to elastomers, foams and rubber. The gasket 404 is an exemplary embodiment to accomplish the goal of sealing or waterproofing the electronics assembly 304. Other embodiments may employ various techniques or materials such as, but not limited to adhesives or physical bonding such as ultrasonic welding or heat staking, to accomplish sealing and/or waterproofing of the electronics assembly 304

In many embodiments the vent 422 is an opening in the housing 310 that passes from an exterior of the housing 310 to an interior (not shown). In many embodiments the vent 422 enables gas from the interior of the housing 310 to be vented into the receiver portion 316. Venting the interior of the housing 310 enables pressure equalization between the interior of the housing 310 and the ambient environment. The barrier 424 is positioned or located within the interior of the housing 310 and prevents moisture from entering or passing through the vent 422. Accordingly, in many embodiments, the barrier 424 is permeable to gasses but impermeable to liquids. An exemplary, non-limiting material for the barrier 424 is a polytetrafluoroethylene membrane.

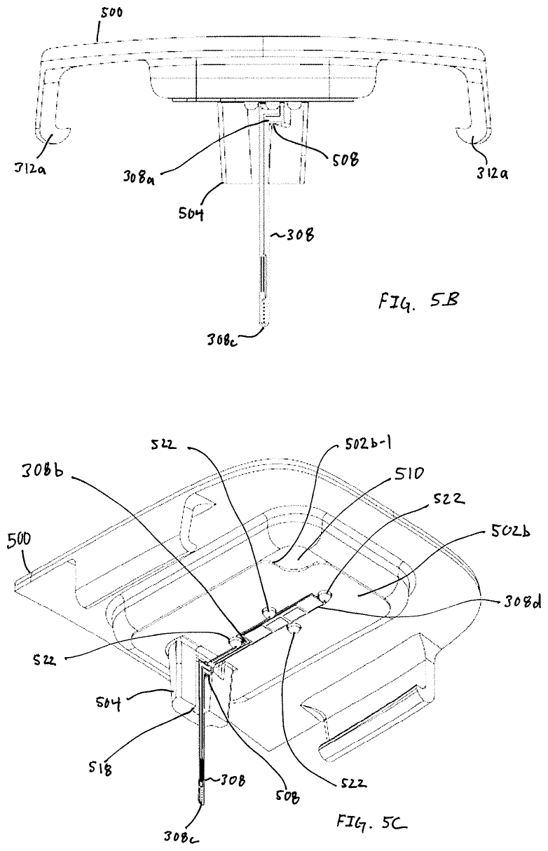

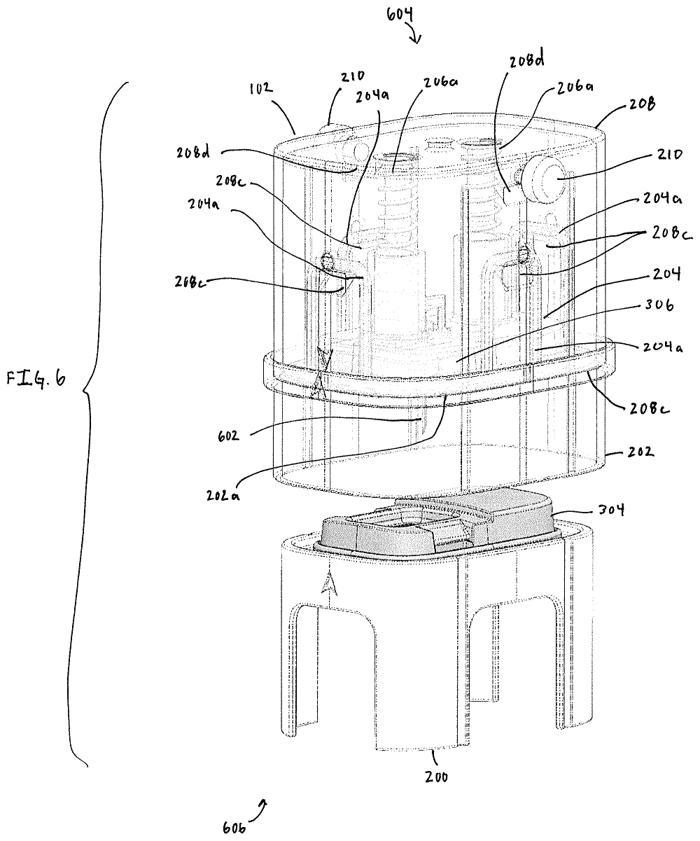

FIG. 5A is an exemplary pseudo-isometric exploded view of the sensor assembly 306, in accordance with embodiments of the present invention. The sensor assembly 306 includes the sensor 308, gaskets 502a and 502b, cap 500 and sensor assembly engagement 506. Cap 500 includes an interior face 510, an exterior face 512 and a cap aperture 526. The cap aperture 526 connects the interior face 510 and the exterior face 512. Moreover, the cap aperture 526 is intended to enable a sharp (not shown) and the sensor 308 to pass through the cap 500. The interior face 510 of the cap 500 is intended to mate with the receiver portion 316 (see FIG. 4). Extending away from the interior face 510 are sensor retainers 522. Sensor retainers 522 are intended to provide guidance or assist in locating or physically positioning the sensor 308 relative to the cap. As illustrated in FIG. 5, the sensor retainers 522 may be boss features that extend away from the interior face 510.

Also extending away from the interior face 500 is a sharp support 504. The sharp support 504 is a structure partially surrounding the cap aperture 526 is configured to physically support the sharp and sensor 308 during an insertion procedure or process. The sharp support includes a channel 518 that is sized to accommodate the sharp and the sensor. The sharp support 504 further includes a sensor support 508. As illustrated, the sensor support 508 is a channel that is offset from the channel 518. The sensor support 508 is intended to interface or accommodate a sensor jog (not shown). The sensor support 508 prevents the sensor 308 from being able to be vertically displaced. Accordingly, the interplay or interaction between the sensor jog and the sensor support 508 ensures the sensor is not inserted past a predetermined depth within a subject.

The sensor 308 includes a plurality of electrical contacts 524. Locating the sensor 308 between the gaskets 502a and 502b is intended to minimize the likelihood of moisture ingress around the electrical contacts 524. Gasket 502a includes a contact opening 514 along with support opening 516a. The contact opening 514 is intended to provide access to the electrical contact 524. Specifically, the contact opening 514 enables the conductor 406 (see FIG. 4) to make contact with the electrical contacts 524 of the sensor assembly 306. The support opening 516a accommodates the sharp support 504. Gasket 502b includes a support opening 516b along with apertures 520. The support opening 516b accommodates the sharp support 504 while the apertures 520 are configured to be aligned with the sensor retainers 522.

In preferred embodiments the gaskets 502a or 502b are intended to function as seals with the intended purpose being to prevent moisture from reaching the sensor 308. Gaskets 502a and 502b further include vent notch 502a-1 and 502b-1, respectively. The vent notches 502a-1 and 502b-1 are located to enable the vent 422 (FIG. 4) to function when the sensor assembly 306 is coupled with the electronics assembly 304 (FIG. 4). Without vent notches 502a-01 and 502b-1, the gaskets 502a and 502b could prevent gasses within the interior from escaping via the vent. In some embodiments the gaskets 502a and 502b can be various combinations of compliant materials such as, but not limited to, foam, rubber, adhesives or combinations thereof. In still other embodiments, one or more of gaskets 502a and 502b can be replaced by other mechanical sealing techniques such as ultrasonic welding, adhesives, interference fits or the like.

For example, in some embodiments, the gasket 502b is a double sided adhesive layer and the gasket 502a is a compliant material such as rubber. Accordingly, in some embodiments, the gasket 502b is coupled to the interior face 510. Moreover, both the gasket 502a and the sensor 308 may be coupled to the gasket 502b. In other embodiments, various combinations of adhesives and other mechanical coupling techniques may be used to secure the sensor 308 between the gaskets 502a and 502b, along with securing the gasket 502b to the cap 500. In preferred embodiments, the sensor retainers 522 extend away from the interior face 510 enough to further extend through the apertures 520 and enable locating the sensor 308 relative to the gasket 502b and the cap 500. The sensor assembly engagement 506 has an engagement top 528 and an engagement bottom 530. In many embodiments the engagement bottom 530 is mated or secured to the exterior face 512 of the cap 500. In preferred embodiments an adhesive secures the sensor assembly engagement 506 to the cap 500. In alternative embodiments, other mechanical fastening techniques may be used to affix the assembly engagement 506 to the cap 500.

FIGS. 5B and 5C are exemplary illustrations of select components within the sensor assembly 306 that illustrate the interaction between the sensor 308 and features of the cap 500, in accordance with embodiments of the present invention. For example, in FIGS. 5B and 5C the sensor 308 is illustrated in an exemplary position or location relative to the cap 500 that is at least partially accomplished with the assistance of at least one feature, or features, associated with the sensor and at least one or more of the sensor support 508 and the sensor retainers 522.

The sensor 308 has a distal end 308c and a proximal end 308d. In preferred embodiments, the distal end 308c is inserted within a subject while the proximal end 308d is retained within the sensor assembly 306. In many embodiments the sensor 308 is a multilayer structure that includes at least one electrical conductor. In many embodiments the electrical conductor for the sensor 308 is selected based on mechanical properties such as, but not limited to, flexibility, ductility, conductivity, corrosion resistance, and toughness. In preferred embodiments, the sensor 308 is made from stainless steel. Use of stainless steel within the sensor 308 enables the sensor 308 to include a contiguous conductor that can further be deformed into the shape illustrated in FIG. 5C. As illustrated, the proximal end 308d is substantially parallel to the interior face 510 while the distal end 308c is substantially normal to the interior face 510.

The sensor 308 includes a first jog 308a that assists in locating the sensor 308 by interacting with the sensor support 508. In many embodiments, the sensor support 508 fits within the first jog 308a, and minimizes the likelihood of the sensor 308 from being pulled through the channel 518 during an insertion process. The sensor support 508 mates or aligns with the first jog 308a to prevent the distal end 308c from being placed deeper than intended during the insertion process.

The channel 518 and the placement of the sensor 308 within the channel 518, restricts potential displacement of the sensor 308. Moreover, the channel being a part of the overall sharp support 504 further aligns the sensor 308 for placement through the support openings 516a and 516b (FIG. 5A), along with the sharp support aperture 410 (FIG. 4), and the base aperture 418 (FIG. 4).

The sensor 308 further includes a second jog 308b that interacts with at least one sensor retainer 522. In many embodiments, the second jog 308b interacts with the sensor retainer 522 to assist in aligning the proximal end 308d relative to the cap 500. Additionally, the sensor retainer 522 that interacts with the second jog 308b further prevents the sensor 308 from being displaced toward the sharp support 504 during an insertion process. Accordingly, as illustrated in FIGS. 5A-5C, the sensor 308 may be retained within the sensor assembly 306 via the gasket 502b, the first jog 308a interacting with the sensor support 508 and the second jog 308b interacting with a sensor retainer 522. The use of the three retention features discussed above should not be construed as limiting. In various other embodiments one or more, or even additional retention features may be used to retain and align the sensor 308 within the sensor assembly 306.

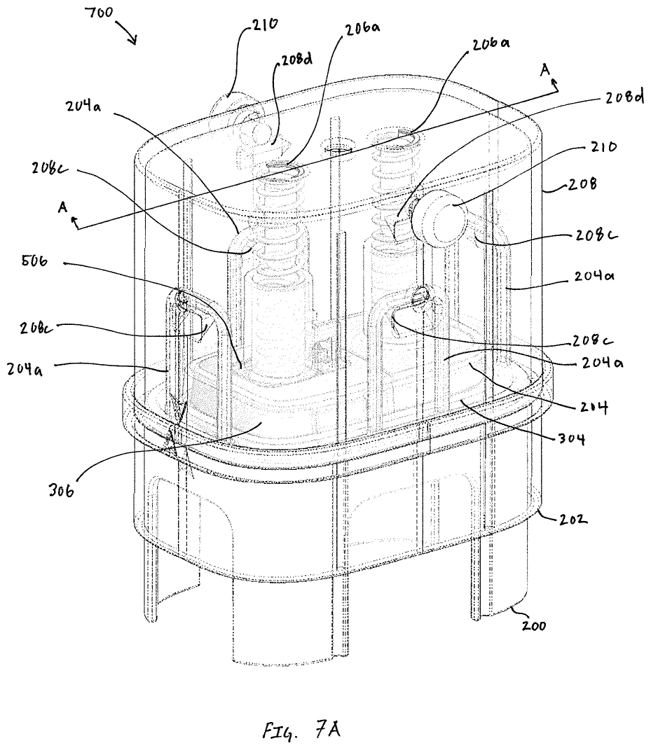

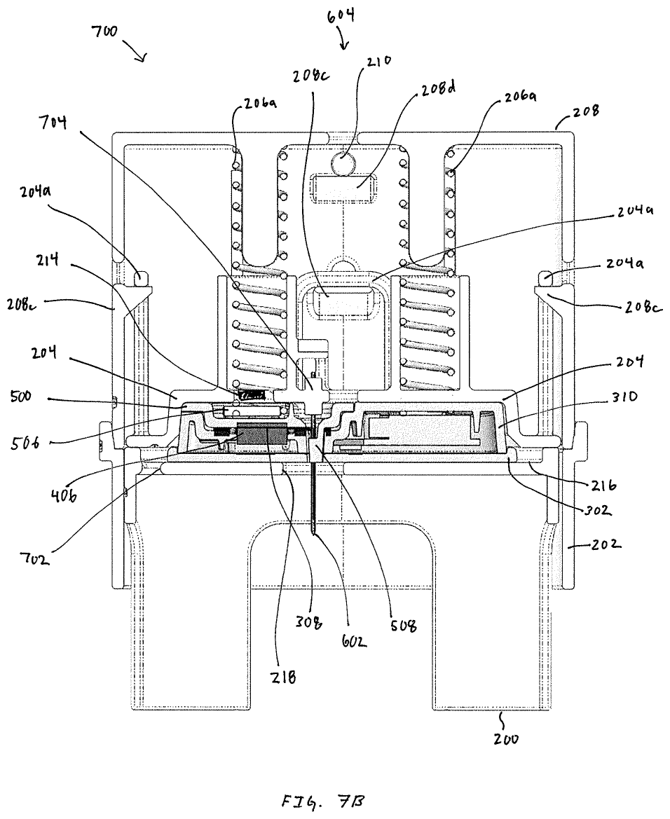

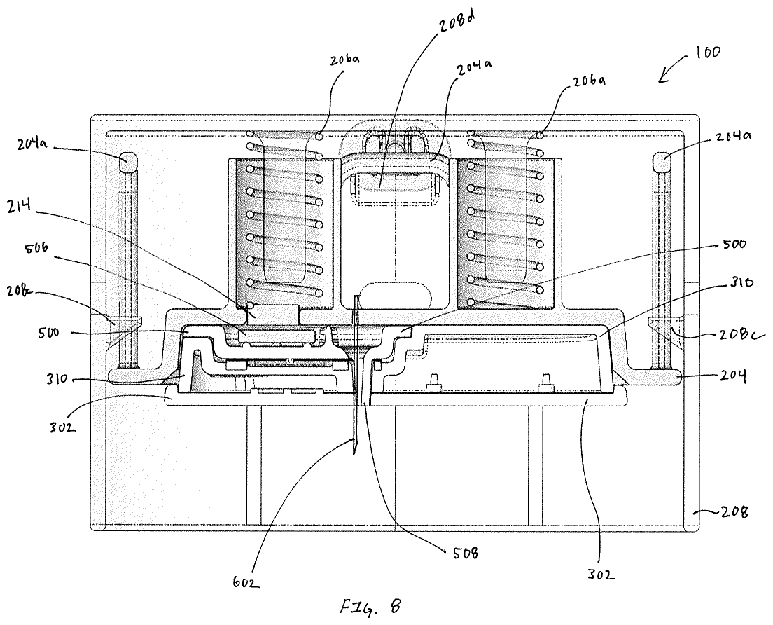

FIG. 6 is an exemplary illustration of the insertion device 102, an electronics assembly 304 (without the patch for clarity) and a sensor assembly 306 positioned relative to each other in a first position that is prior to the sensor assembly 306 being coupled to the electronics assembly 304, in accordance with embodiments of the present invention. It should be noted that FIG. 6 general positions such as a top 604 and a bottom 606 are included and will be used to discuss relative placement or location of features of various components or features. For example, the electronic assembly 304 is located toward the top of the stand 200. FIG. 6 includes an illustration of the sharp 602. In many embodiments, the sharp 602 is anchored, or coupled to the slider 204. In many embodiments the sharp 602 is a hollow needle or channel structure that at least partially surrounds the entirety of the sensor (not shown). The inclusion of the sharp 602 further illustrates that in the first position, the position of the skirt 202 relative to the encasement minimizes exposure to the sharp 602. Additionally, the encasement lip 202a of the skirt is illustrated as being in contact with the encasement bottom 208c. In the first position, energy storage 206a, illustrated as springs, are not compressed or minimally compressed and therefore are not storing any energy or very minimal amounts of energy.

In the first position, the slider engagement 214 (FIG. 2) is removably coupled to the sensor assembly engagement 506 (FIG. 5). The coupling of the slider engagement and the sensor assembly engagement removably mates or couples the sensor assembly 306 to the slider 204. In preferred embodiments, the slider engagement is a magnet and the sensor assembly engagement is a piece of ferrous metal that is attracted to the magnet. In other embodiments, the sensor assembly engagement is a magnet and the slider engagement is a piece of ferrous metal that is attracted to the magnet. In still other embodiments, both the slider engagement 214 and the sensor assembly engagement are magnets positioned to attract each other. In other alternative embodiments, the slider engagement and the sensor assembly engagement are detachably coupled together using other techniques such as, but not limited to, electromagnets, friction fit, suction and the like. Regardless of embodiment, the location or position of the slider engagement 214 within the slider 204 further positions the sensor assembly 306 relative to the electronics assembly 304 so the mating features 312a and 312b are aligned to couple the sensor assembly 306 to the electronics assembly 304. Moreover, the coupling of the sensor assembly 306 with the slider 204 places the sharp 602 and sharp support 508 (FIG. 5) axially in-line with the sharp support aperture 410 (FIG. 4) ensuring that the sharp 602 and sensor 308 (FIG. 5) have an unobstructed path through the bottom of the electronics assembly 304.

As illustrated in FIG. 6, the slider 204 is in the first position with the slide retainers 204a engaged with the first slide stops 208c. Within the encasement 208 is the slider 204 having the sharp 602 and coupled to the slider 204 is the sensor assembly 306 having the sensor (not shown), the sensor being nested within the sharp 602.

FIG. 7A is a pseudo-isometric view of an insertion assembly 700 just after coupling the sensor assembly 306 to the electronics assembly 304 (without the patch for clarity), in accordance with embodiments of the present invention. FIG. 7B is a cross-sectional view of the insertion assembly 700 looking at a section defined by section line A-A in FIG. 7A, in accordance with embodiments of the present invention. FIGS. 7A and 7B, are illustrated having the sensor assembly 308 and the electronics assembly 304 coupled together while it appears that energy storage 206a is uncompressed, or not storing energy. In preferred embodiments the coupling of the sensor assembly 306 and the electronics assembly 304 occurs during the loading of the energy storage 206a. For example, when energy storage 206a is being compressed, the electronics assembly 304 is coupled to the sensor assembly 306 via the mating features 312a and 312b (FIG. 3B). In both FIGS. 7A and 7B, the encasement 308 contains the slider 204 that is coupled with the sensor assembly 306 that is now coupled with the electronics assembly 304 to create the on body assembly 104 (FIG. 3A). FIG. 7B illustrates how first stand aperture 218 enables the sharp 602 and sensor (nested within the sharp 602) to protrude below the depression 216 of the stand. Further visible in FIG. 7B is that the sharp 602 does not extend beyond the skirt 202.

FIG. 7B further includes illustrations of a sharp holder 704 and a second stand aperture 702. The second stand aperture 702 is intended to accommodate the tail 300c (FIG. 3C) of the patch 300 (FIG. 3C) that would normally be attached to the base 302. In many embodiments, the tail is placed through the second stand aperture 702 to assist in placing or locating the base 302 (and attached housing 310) within the depression 216 of the stand 200 via the attached patch 300 (FIG. 3A). The sharp holder 704 is part of the slider 204 is configured to accommodate the sharp 602. Placement of the sharp holder 704 on the slide 204 enables the sharp 602 to at least partially enclose or envelop the sensor during insertion.

FIGS. 7A and 7B further illustrate various interactions between components of the sensor assembly 306 and the electronics assembly 304. For example, in FIG. 7B, the sensor engagement 506 is shown within the cap 500 placed relative to the slider engagement 214 within the slider 204. As previously discussed, in many embodiments the slider engagement 214 and the sensor engagement 506 can be various combinations of a magnet and ferrous metal thereby ensuring that the sensor assembly 306 is initially coupled to the slider 204. Moreover, once the sensor assembly 306 is coupled to the electronics assembly 304 to create the on body assembly 104 shown in FIGS. 7A and 7B, the slider engagement 214 and sensor engagement 506 continue to couple the electronics assembly 304 to the slider 204.

With the stand 200 supported by a surface, when downward pressure is applied to the top 604 of the encasement 208, the top 604 of the encasement 208 will be displaced toward the depression 216 of the stand 200 thereby compressing energy storage 206a. Meanwhile, the slider 204 and on body assembly 104 (FIG. 3A), supported by the stand 200 remain stationary. Continuing to apply downward pressure to the top 604 will eventually displace the encasement 208 enough so that at least some of the slide retainers 204a engage the second slide stop 208d. A second position is defined when at least some of the slide retainers 204a engage the second slide stop 208d. In the second position, the energy storage 206a is storing energy and the actuator 210 are capable of displacing the slide retainers 204a that are engaged with the second slide stop 208d to release the slider 204 so the stored energy in the energy storage 206a can return the slider 204 to the first position.

As downward pressure is applied to the encasement, the conductor 406, protruding from the contact aperture 408 (FIG. 4) makes contact with the sensor 308. In some embodiments, this enables electrical power to be applied to the sensor 308 and instantaneously initiates the sensor 308. In still other embodiments, when the conductor 406 contacts the sensor 308, electronics within the housing 310 that include a logic processor, begin a countdown before initiating the sensor 308. Thus, in some embodiments it is possible for the sensor 308 to be initiated before the sensor is inserted into a subject. In other embodiments, depending on how much time must elapse before the sensor is initiated, the sensor may be initiated after insertion.

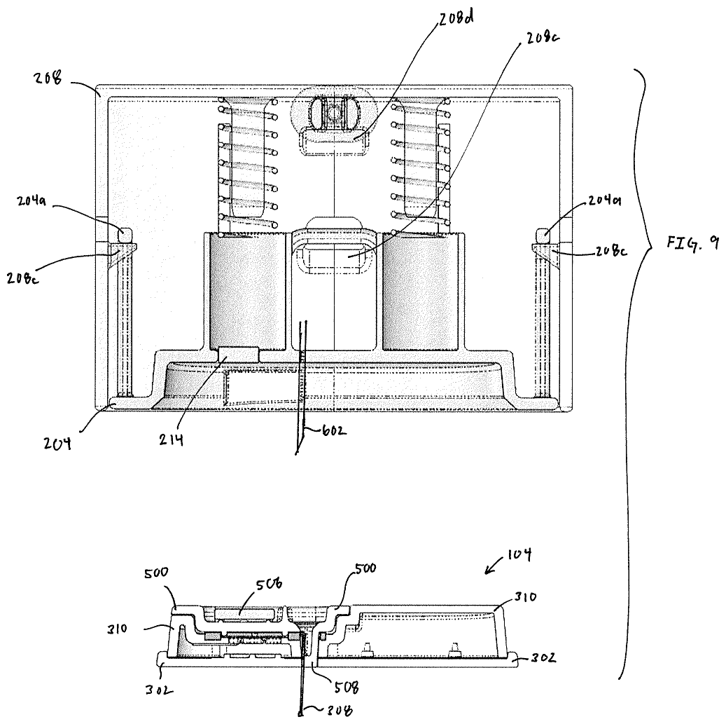

FIG. 8 is a cross-section view of the partial insertion assembly 100 in the second position, in accordance with embodiments of the present invention, and for simplicity, some elements previously illustrated in FIG. 7B have been removed. In FIG. 8, at least one slider retainer 204a is engaged with the second slide stop 208d. Additionally, in FIG. 8, at least two slider retainers 204a are illustrated as being disengaged, or positioned away from, first slide stop 208c. Note that in the second position illustrated in FIG. 8, the sharp 602 does not extend beyond the encasement 208.

Additionally, FIG. 8 provides an illustration of how the sensor support 508, interacts with the electronics assembly 304 and the base 302. Specifically, the cross-section illustrates how the sensor support 508 enables alignment of the sensor assembly 306 and the electronics assembly 304, including the base 302. The precise alignment of these elements enables consistent and repeatable placement of the sensor within the on body assembly 104.

Moreover, FIG. 8 further illustrates aspects of the interaction between the slider 204, the sensor assembly 306 and the electronics assembly 304. For example, the cross-section illustrates the location of the slider engagement 214 within the slider 204. Additionally, the sensor engagement 506 element is illustrated as being coupled to the exterior face of the cap 500 of the sensor assembly 306. As previously disclosed, together the sensor engagement 506 and the slider engagement 214 detachably couple the sensor assembly 306 to the slider 204. The coupling of the sensor assembly 306 with the slider 204 aligns the sensor 308 (FIG. 7B) with the sharp 602. The alignment of the sensor with the sharp 602 is necessary to ensure the subsequent alignment of the sensor assembly 306 with the electronics assembly 304 and the base 302 via the sensor support 508, discussed above.

When engaged in the second position illustrated in FIG. 8, energy storage 206a is actively storing potential energy that upon being released, enables the slider 204 to rapidly or quickly return to a position with lower potential energy, for example, the first position; thereby inserting the sensor into a subject along with the needle. In many embodiments, including FIG. 8, energy storage 206a is a plurality of coiled springs. However, in other embodiments, alternative energy storage 206a may be used including the use of fewer, more, or combinations of energy storage devices or mechanisms. While in the second position and separated from both the stand and the skirt, as illustrated in FIG. 8, in many embodiments the liner is removed from the patch 300 (FIG. 3A-3C) to expose an adhesive. The purpose of the adhesive on the bottom of the patch is to mechanically secure the on body assembly 104 (the sensor assembly 306 coupled to the electronics assembly) to a subject.

FIG. 9 is a cross-section view of the partial insertion assembly 100 after the slider 204 has returned to the first position and the on body assembly 104 has been detached or decoupled from the slider 204, in accordance with embodiments of the present invention. After returning to the first position, as indicated by the slider retainers 204a being engaged with the first slide stop 208c, the on body assembly 104 may be decoupled from the slider 204. In preferred embodiments, the patch 300 (not illustrated, but found in FIGS. 3A-3C, 4) secures the on body assembly 104 to a subject using an adhesive disposed on the bottom of the patch 300.

In many embodiments, the force coupling the slider engagement 214 to the sensor engagement may be less than the force that couples the patch adhesive of the on body assembly 104 to the subject. Accordingly, the encasement 208 (and slider 204 retained within the encasement 208) may be pulled away from the subject decoupling the on body assembly 104 from the slider 204. In other embodiments, the force coupling the slider engagement 214 to the sensor engagement may be more than the force that couples the patch adhesive of the on body assembly 104 to the subject. In these embodiments it may be necessary to use an ejector mechanism or feature to manually or automatically decouple the slider 204 from the on body assembly 104.

As illustrated, in many embodiments, the sharp 602 is retained by the slider 204 after the on body assembly 104 is decoupled from the slider 204. Thus, as the encasement 208 is withdrawn, the sharp 602 may be cleanly withdrawn through the on body assembly 104 while leaving the sensor 308 within the subject.

In many embodiments, additional features or elements can be included, added or substituted for some or all of the exemplary features described above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. An insertion assembly, comprising:
an electronics assembly being supported by a stand, the electronics assembly including a base and a housing, the housing having an enclosed portion and a mating portion;
a sensor assembly being detached from the electronics assembly including a sensor and at least one mating feature, the sensor assembly being detachably coupled to a slider in a first position, the slider being coupled to an energy storage system;
wherein the slider is configured to transition to a second position that couples the sensor assembly to the mating portion to define an on-body assembly, the transition to the second position loads the energy storage system and upon actuation, the energy storage system is unloaded to transition the slider to the first position and the on-body assembly is positioned to decouple from the slider.

2. The insertion assembly as described in claim 1, further comprising:
a slider engagement coupled with the slider
a sharp being coupled with the slider; and
a patch coupled to the base, the patch having a skin-side adhesive to couple the on-body assembly to a subject.

3. The insertion assembly as described in claim 2, wherein a first coupling force between the skin-side adhesive and the subject is greater than a second coupling force between the on-body assembly and the slider.

4. The insertion assembly as described in claim 1, wherein the electronics assembly further includes electrical circuitry along with a power supply being contained within the enclosed portion and a conductor that extends from within the housing into the mating portion.

5. The insertion assembly as described in claim 4, wherein the sensor assembly further includes a cap that includes a cap feature, the cap feature having an interior face and an exterior face.

6. The insertion assembly as described in claim 5, wherein the sensor has a distal end and a proximal end, the proximal end being parallel with the interior face.

7. The insertion assembly as described in claim 6, wherein the cap further includes a sharp support that extends away from the interior face, the sharp support further having a channel.

8. The insertion assembly as described in claim 7, wherein the sharp support further includes a sensor support being formed within the channel.

9. The insertion assembly as described in claim 8, wherein the sensor further includes a first jog, the first jog interacting with the sensor support to position the sensor within the channel.

10. The insertion assembly as described in claim 9, wherein the cap includes at least one sensor retainer being formed on the interior face of the cap.

11. The insertion assembly as described in claim 10, wherein the sensor further includes a second jog, the second jog interacting with the sensor retainer to position the sensor relative to the cap.

12. The insertion assembly as described in claim 6, further including a first gasket being coupled to the interior face of the cap.

13. The insertion assembly as described in claim 12, wherein the proximal end of the sensor is coupled to the first gasket.

14. The insertion assembly as described in claim 7, wherein the distal end extends away from the channel.

15. The insertion assembly as described in claim 5, wherein the sensor receives power from the power supply upon coupling of the electronics assembly and the sensor assembly.

16. The insertion assembly as described in claim 13, further including a second gasket being coupled to both the sensor and the first gasket.

17. The insertion assembly as described in claim 16, wherein the second gasket includes a contact opening, the contact opening exposing electrical contact of the sensor.

18. The insertion assembly as described in claim 17, wherein the electronics assembly includes a vent being formed in a receiver portion.

19. The insertion assembly as described in claim 14, wherein the electronics assembly includes a sharp support aperture the sharp support aligns the sharp and sensor to

20. The insertion assembly as described in claim 19, wherein the sharp support fits within the sharp support aperture to place the sharp and sensor to pass through the electronics assembly.
